(19) United States
(12) Patent Application Publication
(10) Pub. No.: US 2017/0045499 A1
(43) Pub. Date: Feb. 16, 2017

(54) NOCICEPTOR-LIKE CELLS DIFFERENTIATED FROM HUMAN NEURAL PROGENITORS AND USES THEREOF

(71) Applicant: University of Central Florida Research Foundation, Inc., Orlando, FL (US)

(72) Inventors: James J. Hickman, Orlando, FL (US); Xiufang Guo, Orlando, FL (US)

(21) Appl. No.: 15/235,867

(22) Filed: Aug. 12, 2016

Related U.S. Application Data
(60) Provisional application No. 62/205,424, filed on Aug. 14, 2015.

(51) Int. Cl. G01N 33/50; C12N 5/0793

(52) U.S. Cl. CPC G01N 33/5058; C12N 5/062; C12N 2500/90; C12N 2501/13; C12N 2500/46; C12N 2502/09; C12N 2533/54

(57) ABSTRACT

Disclosed herein are methods of differentiating human neural progenitor cells to nociceptor-like cells. Also disclosed are methods of making an innervated skin-like construct using nociceptor-like cells differentiated from human neural progenitor cells. Also disclosed are engineered constructs for screening potentially therapeutic compounds that include a skin-like construct and nociceptor-like cells differentiated from human neural progenitor cells. Also disclosed is a method of screening potential therapies.

### CROSS-REFERENCE TO RELATED APPLICATIONS

This application claims the benefit of U.S. Provisional Application No. 62/205,424, filed Aug. 14, 2015, which is hereby incorporated herein by reference in its entirety.

### STATEMENT REGARDING FEDERALLY SPONSORED RESEARCH OR DEVELOPMENT

This invention was made with Government Support under Grant No. NIH UH2TR000516 awarded by the National Institutes of Health of the United States of America. The Government has certain rights in the invention.

### BACKGROUND

Sensory neurons are responsible for conveying internal, external, and environmental stimuli to the central nervous system (CNS). Nociceptors, or pain mediating sensory neurons, are particularly important for detection of damaging stimuli. Sensory neurons act as signal initiators in all reflex responses, and constitute an indispensible component for the correct function of the nervous system. Sensory neurons can be damaged or injured through a variety of means such as traumatic injury, infection, toxin exposure, metabolic disease, immune system disorders, cancer and chemotherapy, and heredity. The subsequent cellular dysfunction caused by such damage or injury is associated with symptoms ranging from abnormal sensation to numbness and pain to loss of coordination in voluntary movement.

In general, culturing embryonic stem cells (ESCs) is a lengthy process that requires the handling of aggregated embryoid bodies and neurospheres. To date, such ESC approaches have only produced small yields of the differentiated cellular phenotypes. In such cultures, neural crest cells are typically found interspersed with neural rosettes, and therefore, cellsorting is required to obtain a highly enriched cell population. The generation of nociceptors from ESCs requires a series of stages: (1) the induction of neural ectoderm, which distinguishes the developing nervous system from other systems; (2) the induction of neural crest cell fate, which segregates the peripheral nervous system (PNS) from the CNS, and (3) the differentiation of sensory neurons, which distinguishes them from other neural crest derivatives, and then the differentiation of nociceptors from sensory neurons. Currently available culturing protocols lack the ability to generate a population of functional nociceptors neurons without employing a series of technically challenging, time-consuming, and contaminating steps.

Therefore, there is still a scarcity of compositions, methods, and kits that effectively provide an in vitro source of human (and non-human) nociceptors, which would generate invaluable material for use in functional human (and non-human) disease models, in pathological studies and drug screening, and in regenerative medicine. These needs and other needs are satisfied by the compositions, methods, and kits disclosed herein.

### SUMMARY

Disclosed herein are methods of differentiating human neural progenitor cells to nociceptor-like cells. The methods include exposing human neural progenitor cells to a first serum-free initiation medium for a first time period. The methods can further include exposing the human neural progenitor cells to a second serum-free initiation medium for a second time period. The methods can further include exposing the human neural progenitor cells to a serum-free differentiation medium during a third time period to cause at least a portion of the human neural progenitor cells to differentiate into nociceptor-like cells. These nociceptor-like cells possess at least one nociceptor-like property.

The first serum-free initiation medium can include KSR base medium, SB43152, and LDN-193189. In some aspects, the concentration of SB43152 in the first serum-free initiation medium is from 1-100 μM, 1-50 μM, 1-20 μM, or 5-10 μM. In some aspects, the SB43152 concentration in the first-serum free initiation medium is about 10 μM. In some aspects, the concentration of LDN-193189 in the first-serum free initiation medium is from 10-1000 nM, 10-500 nM, 10-300 nM, 50-200 nM. In some aspects, the concentration of LDN-193189 in the first-serum free initiation medium is about 100 nM. In some aspects, the first time period is from 1-5 days. In some aspects, the first time period is about 2 days.

The second serum-free initiation medium can include SB43152, LDN-193189, CHIR99021, and DAPT. In some aspects, the concentration of SB43152 in the second serum-free initiation medium is from 0.8-80 μM, 0.8 to 40 μM, 1-20 μM, 5-10 μM. In some aspects, the concentration of SB43152 in the second serum-free initiation medium is about 8 μM. In some aspects, the concentration of LDN-193189 in the second serum-free initiation medium is from 10-1000 nM, 10-500 nM, 20-300 nM, 50-200 nM. In some aspects, the concentration of LDN-193189 in the second serum-free initiation medium is about 100 nM. In some aspects, the concentration of CHIR99021 in the second serum-free initiation medium from 0.3-30 μM, 0.3-10 μM, 15-30 μM, 1-10 μM, 2-5 μM. In some aspects, the concentration of CHIR99021 in the second serum-free initiation medium about 3 μM. In some aspects, the concentration of DAPT in the second serum free initiation medium is from 0.4-40 μM, 0.4-20 μM, 1-10 μM, 2-8 μM. In some aspects, wherein the concentration of DAPT in the second serum free initiation medium is about 4 μM.

The second serum-free initiation medium further can also include KSR base medium, N2B base medium, or a combination thereof. The methods disclosed herein may include gradually reducing a ratio of KSR base medium to N2B base medium during the second time period. The second time period can be from 9-15 days. In some aspects, the second time period is about 10 days.

The serum-free differentiation medium can include N2B base medium, BDNF, GDNF, and NGF. In some aspects, the concentration of BDNF in the serum-free differentiation medium can be from 2.5-250 ng/mL, 5-100 ng/mL, 10-50 ng/mL. In some aspects, the concentration of GDNF in the serum-free differentiation medium can be from 2.5-250 ng/mL, 5-100 ng/mL, 10-50 ng/mL. In some aspects, the concentration of NGF in the serum-free differentiation medium can be from 2. 2.5-250 ng/mL, 5-100 ng/mL, 10-50 ng/mL. In some aspects, the concentration of BDNF in the serum-free differentiation medium is about 25 ng/mL, the concentration of GDNF in the serum-free differentiation medium is about 25 ng/mL, and the concentration of NGF in the serum-free differentiation medium is about 25 ng/mL. In some aspects, the third time period is at least two days.

The human neural progenitor cells may be in contact with surface comprising an aminated alkylsilane during the first time period, the second time period, the third time period, or any combination of the above. In some aspects, the aminated alkylsilane is DETA.

The nociceptor-like property may include expression of Runx1, TrkA, Ret, IB4, CGRP or a combination thereof. The nociceptor-like property may include expression of an mRNA transcript partially or fully encoding the proteins Runx1, TrkA, Ret, IB4 and/or CGRP.

Disclosed herein is a method of making an innervated tissue construct, e.g. skin-like construct. The method can include differentiating human neural progenitor cells to nociceptor-like cells, contacting the nociceptor-like cells with a tissue construct, and incubating the nociceptor-like cells and the tissue construct under conditions suitable to induce axon extension from the nociceptor-like cells into the tissue construct.

Disclosed herein is an engineered construct for screening potentially therapeutic compounds. The construct includes a tissue construct and nociceptor-like cells differentiated from human neural progenitor cells. The nociceptor-like cells include axons extending from the nociceptor-like cells into the tissue construct. Also disclosed is a method of screening potential therapies. The method includes exposing the engineered construct to a candidate therapy, and measuring a response from at least one nociceptor-like cell within the tissue construct.

### DESCRIPTION OF DRAWINGS

FIG. 1 shows phase contrast pictures demonstrating the morphological change of the cells at days 0, 2, and 11 of the nociceptor induction.

FIGS. 2A to 2C show immunocytochemistry characterization of differentiated cells indicated the expression of typical nociceptor markers. FIG. 2A shows Runx1 and DAPI staining in a Day 7 culture. The images are taken from the same area of the culture plate/same field of view. FIG. 2B shows Co-immunostaining of Ret and TrkA in a Day 13 culture. FIG. 2C shows Co-immunostaining of TrkA and Peripherin in a Day 28 culture.

FIGS. 3A to 3C show characterization of induced nociceptors with peptidergic marker CGRP and non-peptidergic marker IB4. FIG. 3A shows IB4 staining in a Day 24 live culture. FIG. 3B shows Co-immunostaining of IB4 and TrkA in a Day 47 culture. FIG. 3C shows Co-immunostaining of CGRP and Ret in a D30 culture.

FIGS. 4A and 4B show electrophysiological recording from an induced culture. FIG. 4A shows an action potential recorded from an induced neuron shown as in the inset picture. FIG. 3B shows Inward (INa+) and outward (IK+) currents recorded by voltage-clamp.

FIGS. 5A and 5B show H & E staining of the human skin construct after one week of culture in control medium (FIG. 5A) and serum-free human nociceptor medium (FIG. 5B).

FIGS. 6A and 6B show a 3D trans-well system developed for the co-culture of nociceptors and a skin construct. The skin construct was placed on top of the nociceptors and cultured under a semi-air condition. FIG. 6A shows a Diagram of the co-culture setup. FIG. 6B shows a photograph of the co-culture setup.

FIGS. 7A to 7D show immunostaining of the skin sections with Peripherin after nineteen days co-culture displayed the penetration of sensory axons into the skin tissue. FIG. 7A shows H&E staining. FIGS. 7B to 7D show Immunohistological staining of the skin sections demonstrated the penetration of sensory neuron axons (stained by Peripherin) into the skin.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. For example, if the value “10” is disclosed, then “about 10” is also disclosed. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). The term “subject” can also refer to the source of a material, such as a biological material, for example, cells, tissues, organs, etc.

In an aspect, a subject can be afflicted with one or more diseases or disorders, such as, for example, a CNS (central nervous system) or PNS (peripheral nervous system) disease or disorder. The terms “CNS disease” or “CNS disorder” refer to neurological and/or psychiatric changes in the CNS, e.g., brain and spinal cord, which changes manifest in a variety of symptoms. Examples of CNS diseases or disorders include, but are not limited to, the following: migraine headache; cerebrovascular deficiency; psychoses including paranoia, schizophrenia, attention deficiency, and autism; obsessive/compulsive disorders including anorexia and bulimia; convulsive disorders including epilepsy and withdrawal from addictive substances; cognitive diseases including Parkinson's disease and dementia; and anxiety/depression disorders such as anticipatory anxiety (e.g., prior to surgery, dental work and the like), depression, mania, seasonal affective disorder (SAD); and convulsions and anxiety caused by withdrawal from addictive substances such as opiates, benzodiazepines, nicotine, alcohol, cocaine, and other substances of abuse. Further examples of CNS diseases and disorders include, but are not limited to, the following: Abercrombie's degeneration, Acquired epileptiform aphasia (Landau-Kleffner Syndrome), Acute Disseminated Encephalomyelitis, Adrenoleukodystrophy, Agnosia, Alexander Disease, Alpers' Disease, Alternating Hemiplegia, Amyotrophic Lateral Sclerosis, Angelman Syndrome, Ataxia Telangiectasia, Ataxias and Cerebellar/Spinocerebellar Degeneration, Attention Deficit Disorder, Binswanger's Disease, Canavan Disease, Cerebral Hypoxia, Cerebro-Oculo-Facio-Skeletal Syndrome, Charcot-Marie-Tooth, Chronic Inflammatory Demyelinating Polyneuropathy (CIDP), Corticobasal Degeneration, Creutzfeldt-Jakob disease, Degenerative knee arthritis, Diabetic neuropathy, Early Infantile Epileptic Encephalopathy (Ohtahara Syndrome), Epilepsy, Friedreich's Ataxia, Guillain-Barre Syndrome (GBS), Hallervorden-Spatz Disease, Huntington's Disease, Krabbe Disease, Kugelberg-Welander Disease (Spinal Muscular Atrophy), Leigh's Disease, Lennox-Gastaut Syndrome, Machado-Joseph Disease, Macular degeneration, Monomelic Amyotrophy, Multiple Sclerosis, Neuroacanthocytosis, Niemann-Pick disease, Olivopontocerebellar Atrophy, Paraneoplastic Syndromes, Parkinson's Disease, Pelizaeus-Merzbacher Disease, Pick's Disease, Primary Lateral Sclerosis, Progressive Locomotor Ataxia (Syphilitic Spinal Sclerosis, Tabes Dorsalis), Progressive Supranuclear Palsy, Rasmussen's Encephalitis, Rett Syndrome, Tourette's Syndrome, Usher syndrome, West syndrome (Infantile Spasms), and Wilson Disease. General characteristics of such diseases are known in the art. The skilled person can identify additional CNS diseases and disorders known in the art without undue experimentation.

As used herein, the terms “PNS disease” or “PNS disorder” can refer to a disease, illness, condition, or disorder that affects part or all of the peripheral nervous system. The PNS can comprise all the nerves in your body, aside from the ones in the brain and spinal cord. The PNS can act as a communication relay between the brain and the extremities. Unlike the CNS, the PNS is not protected by bone or the blood-brain barrier, which renders it exposed to toxins and mechanical injuries. Generally, the PNS can be divided into the somatic nervous system and the autonomic nervous system. As known to the art, there are over 100 types of PNS diseases and disorders. The causes of these PNS diseases or disorders include, but are not limited to, the following: diabetes, genetic predispositions (hereditary causes); exposure to toxic chemicals, alcoholism, malnutrition, inflammation (infectious or autoimmune), injury, and nerve compression; and by taking certain medications such as those used to treat cancer and HIV/AIDS. PNS diseases and disorders include anesthesia, hyperesthesia, paresthesia, and neuralgia. PNS diseases and disorders include, but are not limited to, the following: accessory nerve disorder, acrodynia, hand-arm vibration syndrome, amyloid neuropathies, anesthesia dolorosa, anti-mag peripheral neuropathy, autonomic dysreflexia, axillary nerve dysfunction, axillary nerve palsy, brachial plexus neuropathies, carpal tunnel syndrome, Charcot-Marie-Tooth disease, chronic solvent-induced encephalopathy, CMV polyradiculomyelopathy, complex regional pain syndromes, congenital insensitivity to pain with anhidrosis, diabetic neuropathies, dysautonomia, facial nerve paralysis, facial palsy, familial dysautonomia, Guillain-Barre syndrome, hereditary sensory and autonomic neuropathy, Homer's syndrome, Isaacs syndrome, ischiadica, leprosy, mononeuropathies, multiple system atrophy, myasthenia gravis, myotonic dystrophy, nerve compression syndrome, nerve injury, neuralgia, neuritis, neurofibromatosis, orthostatic hypotension, orthostatic intolerance, primary autonomic failure, pain insensitivity (congenital), peripheral nervous system neoplasms, peripheral neuritis, peripheral neuropathy, piriformis syndrome, plexopathy, polyneuropathies, polyneuropathy, postherpetic neuralgia, postural orthostatic tachycardia syndrome, pronator teres syndrome, proximal diabetic neuropathy, pudendal nerve entrapment, pure autonomic failure, radial neuropathy, radiculopathy, sciatica, Tarlov cysts, thoracic outlet syndrome, trigeminal neuralgia, ulnar neuropathy, vegetative-vascular dystonia, Villaret's syndrome, Wartenberg's syndrome, and winged scapula.

As used herein, the term “diagnosed” can mean having been subjected to a physical, psychological, and/or psychiatric examination by a person of skill, such as, for example, a physician, a psychologist, and/or psychiatrist, and found to have a condition that can be diagnosed or treated by the compositions or methods disclosed herein.

As used herein, the term “treatment” can refer to the medical management of a subject with the intent to cure, ameliorate, stabilize, or prevent a disease, pathological condition, or disorder (such as, for example, a CNS or PNS disease or disorder). This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term can cover any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression or amelioration of the disease.

As used herein, the term “candidate treatment” refers to a treatment that is currently being tested for its effectiveness against a disease or disorder.

As used herein, the term “prevent” or “preventing” can refer to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the terms “administering” and “administration” refer to any method of providing a disclosed composition, complex, or a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In an aspect, a preparation can be administered therapeutically; that is, administered to treat an existing disease or condition. In an aspect, a preparation can be administered prophylactically; that is, administered for prevention of a disease or condition.

The terms “exposing” or “contacting” as used herein can refer to bringing a disclosed composition, compound, or complex (such as, for example, one or more of the mediums disclosed herein) together with an intended target (such as, e.g., a cell or population of cells, a cell culture, a receptor, an antigen, or other biological entity) in such a manner that the disclosed composition, compound, or complex can affect the activity of the intended target (e.g., receptor, transcription factor, cell, population of cells, a cell culture, etc.), either directly (i.e., by interacting with the target itself), or indirectly (i.e., by interacting with another molecule, co-factor, factor, or protein on which the activity of the target is dependent). In an aspect, a disclosed composition or a disclosed medium can be contacted with a cell or population of cells, such as, for example, a population of neural progenitor cells or a population of nociceptors. For example, a population of cells, such as neural progenitor cells, can be contacted with a disclosed medium (or disclosed mediums) by submerging the cells in a medium, coating the cells with a medium, dipping the cells in a medium, brushing the cells with a medium, bathing the cells in a medium, washing the cells in a medium. The skilled person is familiar with methods used to contact one or more mediums with cells, a population of cells, and/or a cell culture.

As used herein, the term “determining” can refer to measuring or ascertaining (i) an activity or an event, (ii) a quantity or an amount, (iii) a change in activity or an event, or (iv) a change in a quantity or an amount. Determining can also refer to measuring a change prevalence and/or incidence of an activity, or an event, or a trait, or a characteristic. For example, determining can refer to measuring or ascertaining the level of differentiation of a population of cells. The art is familiar with methods and techniques used to measure or ascertain (i) an activity or an event, (ii) a quantity or an amount, (iii) a change in activity or an event, (iv) a change in a quantity or an amount, or (v) a change in prevalence and/or incidence of an activity, or an event, or a trait, or a characteristic. For example, the art is well versed in the use of immunohistochemistry to identify, characterize, and quantify a particular cell type (e.g., a sensory neuron, a nociceptor, a Schwann cell, a neural crest stem cell).

As used herein, the terms “effective amount” and “amount effective” can refer to an amount that is sufficient to achieve the desired result. For example, in an aspect, an effective amount of a disclosed composition or complex or agent is the amount effective to induce differentiation of a population of cells to a desired cell or a desired population of cells. For example, in an aspect, an amount effective is the amount of a disclosed composition or disclosed medium required to (i) induce differentiation of a population of neural progenitor cells to a nociceptors.

A “therapeutically effective amount” can refer to an amount that is sufficient to achieve the desired therapeutic result or to have an effect on undesired symptoms (such as, for example, symptoms associated with a CNS or PNS disease or disorder), but is generally insufficient to cause adverse side effects. The specific therapeutically effective dose level for any particular subject will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a composition or complex at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”, that is, an amount effective for prevention of a disease or condition, such as a CNS or PNS disease or disorder including, but not limited to, those CNS and PNS diseases and disorders disclosed herein.

“Proliferation medium” can refer to supplemented AB2™ basal medium (Aruna Biomedical, Cat. No. hNP7011.3, see U.S. Pat. No. 6,200,806, which is herein incorporated by reference in its entirety for teachings regarding proliferation medium) comprising bFGF. Supplemented AB2 basal medium comprises ANS™ supplement (Aruna Biomedical, Cat. No. ANS7011.2), LIF, L-Glutamine, and Penicillin/Streptomycin.

“KSR medium” can refer to medium prepared by supplementing Knockout DMEM (Invitrogen, Cat. 11330-032) with components including at least KSR (knockout serum replacement—Invitrogen, Cat. 10828-028), L-glutamine, MEM, and β-mercaptoethanol. In an aspect, KSR medium can be prepared by supplementing 800 ml Knockout DMEM (Invitrogen, Cat. 11330-032) with 150 ml KSR (knockout serum replacement, (Invitrogen, Cat. 10828-028), 10 ml L-Glutamine (Invitrogen, Cat. 21051-016), 10 ml Penicillin/Streptomycin (100×, Invitrogen, Cat. 15070-063), 10 ml 10 mM MEM (100×, nonessential amino acids, Invitrogen, Cat. 11140-050) and 1 ml β-mercaptoethanol (1,000×, Invitrogen, Cat. 21985-023).

“N2B medium” can refer to a medium purchased at NeuralStem Inc. In an aspect, N2B can be equivalent to the N2 medium described in Lee G, et al. Nat Protoc 2010; 5:688-701. In an aspect, N2B medium can comprise distilled H₂O with DMEM/F12 powder, glucose, NaHCO₃, insulin, apotransferrin, sodium selenite, putrescine, and progesterone. In an aspect, N2B medium can comprise distilled H₂O (985 ml) with DMEM/F12 powder, 1.55 g glucose, 2.00 g NaHCO₃, 25 mg insulin, 0.1 g apotransferrin, 30 nM sodium selenite, 100 μM putrescine, and 20 nM progesterone.

“KSR/N2B medium” can refer to medium comprising KSR medium and N2B medium.

“A2B™ basal neural medium” can refer to a medium engineered for the expansion and proliferation of hNP1 cells. A2B™ basal neural medium can allow neural cultures to maintain a substantially stable karyotype over multiple passages.

As used herein, the term “initiation medium” refers to a medium capable of priming cells for differentiation, or for changing into a different cell type. For example, it may be capable of priming a stem or progenitor cell for differentiation into a more mature cell type. In another aspect, it may be capable of priming a mature cell type for changing into another mature cell type.

As used herein, the term “differentiation medium” refers to a medium capable of causing differentiation. For example, it may be capable of causing differentiation of a stem cell, a progenitor cell, or an adult cell into a different cell type.

As used herein, “serum-free” means that the culture medium contains no serum or plasma, although purified or synthetic serum or plasma components (e.g., growth factors) can be provided in the culture in reproducible amounts.

As used herein, “tissue construct” refers to an engineered tissue. A tissue construct may include cells supported by natural or synthetic biomaterials. Alternatively, a tissue construct may include cells that have been organized into a three-dimensional structure by some method other than the natural pathways of that particular tissue's growth and development. The tissue construct may be engineered to include any cell type that is naturally found in the body of a subject.

As used herein, “innervated” means that at least one nerve axon has touched at least the outer surface of the innervated object, construct, or tissue. In some aspects, the nerve axon has penetrated the outer surface of the innervated object, construct, or tissue. In some aspects, the nerve may electrically or chemically communicate with the innervated object, construct, or tissue.

As disclosed herein, “neural progenitor cells” can be human or non-human neural progenitor cells. Neural progenitor cells may be derived from any cell source. For example, neural progenitor cells may be derived from embryonic stem cells, induced pluripotent stem cells, or they may be directly differentiated from a cell type which is not a stem cell. For example, neural progenitor cells can be hNP1 cells. In an aspect, neural progenitor cells may be STEMEZ™hNP1.

As disclosed herein, the term “nociceptor-like cell” includes nociceptors and cells that possess at least one nociceptor-like property. In some aspects, the nociceptor-like property may include expression of the Runx1, TrkA, Ret, IB4, or CGRP proteins, or a combination thereof. Expression of these proteins can be detected by methods using ligands that bind to the given protein. Ligands that bind to a given protein may be naturally or synthetically derived. The ligand may bind to the native form of the protein, or it may bind to a protein after post-translational modifications. For example, the ligand may bind to an amino acid sequence present in the native form of the protein, or it may bind to a phosphorylated or glycosylated form of the protein.

In some aspects, expression of Runx1, TrkA, Ret, IB4, and/or CGRP may be detected by methods using antibodies against the given protein. Methods using antibodies against the protein may include, for example, immunohistochemistry or electrophoretic methods such as Western blotting. The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

In some aspects, the nociceptor-like property may include expression of a mRNA transcript partially or fully encoding the Runx1, TrkA, Ret, IB4, and/or CGRP proteins. Methods of “determining gene expression levels” include methods that quantify levels of gene transcripts as well as methods that determine whether a gene of interest expressed at all. A measured expression level may be expressed as any quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. Exemplary methods for detecting the level of expression of a gene include, but are not limited to, Northern blotting, dot or slot blots, reporter gene matrix, nuclease protection, RT-PCR, microarray profiling, differential display, 2D gel electrophoresis, SELDI-TOF, ICAT, enzyme assay, antibody assay, and MNAzyme-based detection methods, Optionally a gene whose level of expression is to be detected may be amplified, for example by methods that may include one or more of: polymerase chain reaction (PCR), strand displacement amplification (SDA), loop-mediated isothermal amplification (LAMP), rolling circle amplification (RCA), transcription-mediated amplification (TMA), self-sustained sequence replication (3SR), nucleic acid sequence based amplification (NASBA), or reverse transcription polymerase chain reaction (RT-PCR).

A number of suitable high throughput formats exist for evaluating expression patterns and profiles of the disclosed genes. Numerous technological platforms for performing high throughput expression analysis are known. Generally, such methods involve a logical or physical array of either the subject samples, the biomarkers, or both. Common array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384 or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., xMAP® technology from Luminex (Austin, Tex.), the SECTOR® Imager with MULTI-ARRAY® and MULTI-SPOT® technologies from Meso Scale Discovery (Gaithersburg, Md.), the ORCA™ system from Beckman-Coulter, Inc. (Fullerton, Calif.) and the ZYMATE™ systems from Zymark Corporation (Hopkinton, Mass.), miRCURY LNA™ microRNA Arrays (Exiqon, Woburn, Mass.).

Alternatively, a variety of solid phase arrays can favorably be employed to determine expression patterns in the context of the disclosed methods, assays and kits. Exemplary formats include membrane or filter arrays (e.g., nitrocellulose, nylon), pin arrays, and bead arrays (e.g., in a liquid “slurry”). Typically, probes corresponding to nucleic acid or protein reagents that specifically interact with (e.g., hybridize to or bind to) an expression product corresponding to a member of the candidate library, are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, or combinations thereof can all serve as the substrate for a solid phase array.

In one embodiment, the array is a “chip” composed, e.g., of one of the above-specified materials. Polynucleotide probes, e.g., RNA or DNA, such as cDNA, synthetic oligonucleotides, and the like, or binding proteins such as antibodies or antigen-binding fragments or derivatives thereof, that specifically interact with expression products of individual components of the candidate library are affixed to the chip in a logically ordered manner, i.e., in an array. In addition, any molecule with a specific affinity for either the sense or anti-sense sequence of the marker nucleotide sequence (depending on the design of the sample labeling), can be fixed to the array surface without loss of specific affinity for the marker and can be obtained and produced for array production, for example, proteins that specifically recognize the specific nucleic acid sequence of the marker, ribozymes, peptide nucleic acids (PNA), or other chemicals or molecules with specific affinity.

Microarray expression may be detected by scanning the microarray with a variety of laser or CCD-based scanners, and extracting features with numerous software packages, for example, IMAGENE™ (Biodiscovery), Feature Extraction Software (Agilent), SCANLYZE™ (Stanford Univ., Stanford, Calif.), GENEPIX™ (Axon Instruments).

In some aspects, the nociceptor-like property expression of specific ion channels and receptors that respond to noxious stimuli. SCN10A (Sodium channel) is selectively expressed in nociceptor sensory neurons and is TTX-resistant but can be blocked by chemicals as A-803467. Examples of nociceptor-specific receptors include TrpV1 (the ones responding to capsaicin, mimicking hot pepper effect) and P2RX3 (the ones responding to α,β-methylene-ATP, mimicking inflammatory pain).

It is understood that the compositions and mediums disclosed herein have certain functions. Disclosed herein are certain structural requirements for performing the disclosed functions, and it is understood that there are a variety of structures that can perform the same function that are related to the disclosed structures, and that these structures will typically achieve the same result.

Disclosed herein are uses of nociceptors made by a disclosed method of generating nociceptors. In an aspect, a disclosed method can comprise the population of neural progenitor cells. In an aspect, a disclosed method can comprise the population of neural crest stem cells. In an aspect, neural progenitor cells can be human neural progenitor cells. In an aspect, neural progenitor cells can be non-human neural progenitor cells. In an aspect, disclosed nociceptors can be used in functional human (and non-human) disease models. In an aspect, disease models can be in vitro disease models. In an aspect, disclosed nociceptors can be used in pathological studies. In an aspect, disclosed nociceptors can be used in the screening of candidate therapies. In an aspect, disclosed nociceptors can be used in regenerative medicine.

Nociceptors made by a disclosed method can be used in regenerative medicine. For example, in an aspect, nociceptors can be used during implantation into a subject. In an aspect, a subject has been diagnosed with or suffers from a disease or disorder that affects nociceptors. In an aspect, implantation of nociceptors generated by a disclosed method can treat or prevent disease or disorder that affects nociceptors. In an aspect, following implantation of nociceptors generated by a disclosed method, a subject can regain sensation or function or a subject can experience less pain, less numbness, or less discomfort, or a combination thereof.

Nociceptors made by a disclosed method can be used in conjunction with a prosthetic device. For example, in an aspect, nociceptors can be incorporated into a prosthetic device. In an aspect, a prosthetic device can be used by a subject. In an aspect, a subject has been diagnosed with or suffers from a disease or disorder that affects nociceptors. In an aspect, a disclosed prosthetic device comprising nociceptors generated by a disclosed method can assist a subject in performing tasks utilizing fine motor control. In an aspect, a prosthetic device comprising nociceptors generated by a disclosed method can improve or enhance a subject's fine motor control. As known to the art, fine motor control is the coordination of muscles, bones, and nerves to produce small precise movements (e.g., fine motor control can be picking up a small item with the index finger and thumb).

Nociceptors made by a disclosed method can be used to examine or characterize a mechanosensory complex. As known to the art, a vertebrate body has several conserved mechanisms for the transduction of mechanical forces to sensory neural impulses (i.e., mechanotransduction). These mechanisms include groups of mechanoreceptors, mainly cutaneous in nature, that respond to touch, pressure, and vibration as well as proprioceptors sensitive to changes in muscle length, stretch, and tension. The activity of this reflex mechanism assists in coordinating a diverse range of muscular activities including eye movement, respiration, and fine motor control. Both mechanoreceptors and proprioceptors are composed of specialized receptors innervated by a specific type of sensory neuron.

In an aspect, nociceptors made by a disclosed method can be used to examine the role of a mechanosensory complex in the pathology of one or more muscular dystrophies. Muscular dystrophies refer to a group of inherited disorders that can involve muscle weakness and loss of muscle tissue. Muscular dystrophies are progressive in that these conditions worsen over time. Muscular dystrophies include, but are not limited to, the following: Becker muscular dystrophy, Duchenne muscular dystrophy, Emery-Dreifuss muscular dystrophy, Facioscapulohumeral muscular dystrophy, Limb-girdle muscular dystrophy, Myotonia congenita, and Myotonic dystrophy. Symptoms associated with various muscular dystrophies include intellectual disability, muscle weakness, delayed development of muscle motor skills, difficulty using one or more muscle groups, drooling, eyelid drooping, frequent falls, loss of strength in a muscle or group of muscles as an adult, loss in muscle size, and difficulty in walking.

Nociceptors made by a disclosed method can be used in several in vitro platforms or models. For example, in an aspect, nociceptors made by a disclosed method can be used in an in vitro platform to characterize and/or examine one or more diseases or disorders, such as a CNS or PNS disease or disorder, including those diseases and disorders disclosed herein. For example, in an aspect, nociceptors made by a disclosed method can be used in an in vitro platform or model to characterize and/or examine one or more of the following diseases and disorders: peripheral neuropathy, neuropathic pain, peripheral nerve regeneration, leprosy, and spasticity inducing diseases like Parkinson's disease. In an aspect, a disease or disorder can be peripheral neuropathy. In an aspect, a disease or disorder can be neuropathic pain. In an aspect, a disease or disorder can be peripheral nerve regeneration. In an aspect, a disease or disorder can be leprosy. In an aspect, a disease or disorder can be spasticity inducing diseases like Parkinson's disease.

Nociceptors made by a disclosed method can be used in the development of in vitro models of drug screening. In an aspect, drug screening can be used for a disease or disorder that affects nociceptors. In an aspect, drug screening can be used one or more muscular dystrophies. In an aspect, drug screening can be used with peripheral neuropathy, neuropathic pain, peripheral nerve regeneration, leprosy, and spasticity inducing diseases like Parkinson's disease.

Disclosed herein are methods of differentiating neural progenitor cells to nociceptor-like cells. The methods include exposing neural progenitor cells to a first serum-free initiation medium for a first time period. The methods further include exposing the neural progenitor cells to a second serum-free initiation medium for a second time period. The methods further include exposing the neural progenitor cells to a serum-free differentiation medium during a third time period to cause at least a portion of the neural progenitor cells to differentiate into nociceptor-like cells. The nociceptor-like cells possess at least one nociceptor-like property.

In the methods disclosed herein, neural progenitor cells are proliferated in culture. In an aspect, neural progenitor cells are proliferated in culture to a specific passage, for example, any passage number from 6 to 13. In an aspect, the neural progenitor cells may be expanded to passage 9 or 10.

In an aspect, neural progenitor cells may be proliferated on a tissue culture surface with a coating configured to induce cell adhesion. In an aspect, the tissue culture surface may be coated with extracellular matrix molecules, for example, basement membrane proteins. In an aspect, the tissue culture surface may be coated with a mixture of proteins secreted by Engelbreth-Holm-Swarm mouse sarcoma cells, for example, Matrigel®, Geltrex®, or Cultrex® for the proliferation of the neural progenitor cells.

The neural progenitor cells may be maintained in a proliferation medium. In an aspect, the proliferation medium may be AB2™ basal medium containing bFGF. The proliferation medium may comprise from 2-200 ng/mL bFGF. In an aspect, the proliferation medium may comprise 20 ng/mL bFGF. In an aspect, the medium is changed every one, two, or three days. In an aspect, the neural progenitor cells may be proliferated for one to seven days, for example, for three to five days prior to changing from proliferation medium to initiation medium. In an aspect, the neural progenitor cells are 70-100% confluent prior to changing the medium from proliferation medium to initiation medium. In an aspect, the neural progenitor cells are 95-100% confluent prior to changing the medium from proliferation medium to initiation medium.

The methods of differentiating neural progenitor cells to nociceptor-like cells include placing the neural progenitor cells into contact with a surface comprising an aminated alkylsilane. The neural progenitor cells or differentiating neural progenitor cells may be in contact with a surface comprising an aminated alkylsilane during the first time period of the method, the second time period of the method, the third time period of the method, or any combination of the above. In an aspect, the neural progenitor cells are plated onto a tissue culture surface coated with an aminated alkylsilane at a density of 100-700 cells/mm². In an aspect, the neural progenitor cells are plated onto a tissue culture surface coated with an aminated alkylsilane at a density of 400 cells/mm².

In some aspects, the aminated alkylsilane is DETA. The DETA treated surface may be prepared by methods described in Guo, X., et al., Biomaterials, 2013. 34(18):4418-4427, the disclosure of which is hereby incorporated by reference in its entirety. In some aspects, the aminated alkylsilane is applied to a tissue culture material. In some aspects, the tissue culture material is a glass coverslip. In some aspects, the aminated alkylsilane is applied to a tissue culture material in conjunction with other molecules that facilitate cell adhesion. For example, the aminated alkylsilane may be applied in conjunction with extracellular matrix molecules. In an aspect, the aminated alkylsilane can be applied in conjunction with poly-ornithine, laminin, and fibronectin.

In an aspect, the neural progenitor cells are further proliferated while in contact with an aminated alkylsilane, prior to applying an initiation medium. For example, the neural progenitor cells may be proliferated in proliferation medium for 1-6 days, or 2-3 days, while in contact with an aminated alkylsilane, prior to applying an initiation medium. In an aspect, the neural progenitor cells are further proliferated 70-100% confluency while in contact with an aminated alkylsilane, prior to applying an initiation medium. For example, in some aspects the neural progenitor cells are further proliferated to 85-95% confluency while in contact with an aminated alkylsilane, prior to applying an initiation medium.

The methods of differentiating neural progenitor cells to nociceptor-like cells include exposing neural progenitor cells to a first serum-free initiation medium for a first time period. The first serum-free initiation medium can include KSR base medium, SB43152, and LDN-193189. In some aspects, the concentration of SB43152 in the first serum-free initiation medium is from 1-100 μM, 1-50 μM, 1-20 μM, or 5-10 μM. In some aspects, the SB43152 concentration in the first-serum free initiation medium is about 10 μM. In some aspects, the concentration of LDN-193189 in the first-serum free initiation medium is from 10-1000 nM, 10-500 nM, 10-300 nM, 50-200 nM. In some aspects, the concentration of SB43152 and LDN-193189 may vary by 0-20% during the first time period, as compared to the concentration on the first day of the first time period. In some aspects, the concentration of SB43152 and LDN-193189 stays substantially constant throughout the second time period. In some aspects, the concentration of LDN-193189 in the first-serum free initiation medium is about 100 nM. In some aspects, the first time period is from 1-5 days. In some aspects, the first time period is about 2 days.

The methods of differentiating neural progenitor cells to nociceptor-like cells include exposing neural progenitor cells to a second serum-free initiation medium for a second time period. The second serum-free initiation medium can include SB43152, LDN-193189, CHIR99021, and DAPT. In some aspects, the concentration of SB43152 in the second serum-free initiation medium is from 0.8-80 μM, 0.8 to 40 μM, 1-20 μM, 5-10 μM. In some aspects, the concentration of SB43152 in the second serum-free initiation medium is about 8 μM. In some aspects, the concentration of LDN-193189 in the second serum-free initiation medium is from 10-1000 nM, 10-500 nM, 20-300 nM, 50-200 nM. In some aspects, the concentration of LDN-193189 in the second serum-free initiation medium is about 100 nM. In some aspects, the concentration of CHIR99021 in the second serum-free initiation medium from 0.3-30 μM, 0.3-10 μM, 15-30 μM, 1-10 μM, 2-5 μM. In some aspects, the concentration of CHIR99021 in the second serum-free initiation medium about 3 μM. In some aspects, the concentration of DAPT in the second serum free initiation medium is from 0.4-40 μM, 0.4-20 μM, 1-10 μM, 2-8 μM. In some aspects, wherein the concentration of DAPT in the second serum free initiation medium is about 4 μM.

The second serum-free initiation medium further can also include KSR base medium, N2B base medium, or a combination thereof. The second time period can be from 9-15 days. In some aspects, the second time period is about 10 days. The methods disclosed herein may include gradually reducing a ratio of KSR base medium to N2B base medium during the second time period. For example, in some aspects, the concentration of KSR medium is reduced by 10-40% every 1-3 days. In some aspects, the concentration of KSR medium is reduced by 25% every 2 days. In some aspects, the concentration of SB43152, LDN-193189, CHIR99021, and/or DAPT may vary by 0-20% during the second time period, as compared to the concentration on the first day of the second time period. In some aspects, the concentration of SB43152, LDN-193189, CHIR99021, and/or DAPT stays substantially constant throughout the second time period.

The methods of differentiating neural progenitor cells to nociceptor-like cells further include exposing the neural progenitor cells to a serum-free differentiation medium during a third time period to cause at least a portion of the neural progenitor cells to differentiate into nociceptor-like cells possessing at least one nociceptor-like property. In some aspects, the methods may include a gradual introduction to the differentiation medium. For example, in some aspects, starting with the first day of the third time period, 25% to 75% of the medium currently contacting the cells may be replaced by serum-free differentiation medium at each media change. Media changes may take place every one, two, or three days. In some aspects, 50% of the medium currently contacting the cells is replaced by serum-free differentiation medium on the first day of the third time period, and on every second day thereafter.

The serum-free differentiation medium can include N2B base medium, BDNF, GDNF, and NGF. In some aspects, the concentration of BDNF in the serum-free differentiation medium can be from 2.5-250 ng/mL, 5-100 ng/mL, 10-50 ng/mL; the concentration of GDNF in the serum-free differentiation medium can be from 2.5-250 ng/mL, 5-100 ng/mL, 10-50 ng/mL; and the concentration of NGF in the serum-free differentiation medium can be from 2.5-250 ng/mL, 5-100 ng/mL, 10-50 ng/mL. In some aspects, the concentration of BDNF in the serum-free differentiation medium is about 25 ng/mL, the concentration of GDNF in the serum-free differentiation medium is about 25 ng/mL, and the concentration of NGF in the serum-free differentiation medium is about 25 ng/mL. In some aspects, the third time period is at least two days.

The nociceptor-like property expressed by the nociceptor-like cells may include expression of Runx1, TrkA, Ret, IB4, CGRP or a combination thereof. The nociceptor-like property may include expression of a mRNA transcript partially or fully encoding the proteins Runx1, TrkA, Ret, IB4 and/or CGRP.

Disclosed herein is a method of making an innervated tissue construct. The method can include differentiating neural progenitor cells to nociceptor-like cells, contacting the nociceptor-like cells with a tissue construct, and incubating the nociceptor-like cells and the tissue construct under conditions suitable to induce axon extension from the nociceptor-like cells into the tissue construct. In some aspects, the tissue construct is a skin construct that includes skin cells or skin-like cells. The skin or skin-like cells may be derived from, for example, pluripotent stem cells, embryonic stem cells, multipotent stem cells, progenitor cells, mature non-skin cell types, primary harvests or cell lines. The skin cells or skin-like cells may include keratinocytes, squamous cells, Merkel cells, melanocytes, Langerhans cells, basal cells, vascular cells, lymphatic cells, follicular cells sebaceous gland cells, sudoriferous gland cells, a combination thereof, or a combination of skin-like cells that are functionally similar to the aforelisted cells.

In some aspects, the tissue construct includes cell types from any organ that detects pain. For example, the tissue construct may include cells from mucosal tissues, sensory tissues, muscle tissues, respiratory tissues, skeletal tissues, cardiovascular tissues, joint tissues, glandular tissues, or gastrointestinal or digestive tissues.

Tissue constructs may be made, for example, by integrating cells of the particular tissue type into a cytocompatible material. As used herein, cytocompatible means non-toxic to the particular cell type. In some examples, the cytocompatible material may be hydrogel, and the hydrogel may be gelled around the cells of the particular tissue type. The hydrogel may include natural or synthetic polymer materials. In some aspects, the hydrogel may include extracellular matrix materials. Cells of a particular tissue type may also be seeded onto and into a pre-formed, cytocompatible scaffold structure to form a tissue construct. Tissue constructs may also be formed by layering cell sheets. The cell sheets may be layered directly onto one another, or cytocompatible materials may be present between the cell sheets.

Some aspects of the method of making an innervated tissue construct may also include suspending the nociceptor-like cells in a hydrogel. The hydrogel may include natural or synthetic polymer materials. In some aspects, the hydrogel may include extracellular matrix materials. For example, the hydrogel may be a mixture of collagen I and Matrigel in DPBS. In some aspects, the hydrogel may be a mixture of 1.5 mg/mL collagen I and 1 mg/mL Matrigel in DPBS. The nociceptor-like cells may be suspended in the hydrogel at a cell density of 0.2×10⁶ to 5×10⁶ cells/mL. For example, in some aspects, the nociceptor-like cells may be suspended in the hydrogel at a cell density of 1.5×10⁶ cells/mL. The hydrogel is then gelled around the nociceptor-like cells, encapsulating the cells therein.

Some aspects of the method of making an innervated tissue construct may also include placing the hydrogel containing the nociceptor-like cells may into contact with the tissue construct. In some aspects, the hydrogel is placed into contact with the tissue construct when it is partially gelled. In some aspects, the hydrogel is placed into contact with the tissue construct when it is fully gelled.

In some aspects of the method of making an innervated tissue construct, the tissue construct is placed on top of the hydrogel containing the nociceptor-like cells. In some aspects, medium is added to submerge only part of the hydrogel. In some aspects, medium is added to submerge all of the hydrogel, but none of the tissue construct. In some aspects, medium is added to submerge all of the hydrogel, and part of the tissue construct. In some aspects, medium is added to submerge all of the hydrogel, and all of the tissue construct. In some aspects, the medium is serum-free differentiation medium. In some aspects, the medium is changed every 1, 2, or 3 days.

Disclosed herein is an engineered construct for screening potentially therapeutic compounds. The construct includes a tissue construct and nociceptor-like cells differentiated from neural progenitor cells. The nociceptor-like cells include axons extending from the nociceptor-like cells into the tissue construct. Also disclosed is a method of screening candidate therapies. The method includes exposing the engineered construct to a candidate therapy, and measuring a response from at least one nociceptor-like cell within the tissue construct. In some aspects, the tissue construct is a skin construct.

### EXAMPLES

### Example 1

### Induction of Nociceptors from Human Neural Progenitors and their Innervation of Human Skin

Sensory innervation is an important elements for skin function. Nociceptors, the sensors for various damaging stimuli, mediate the vital perception—pain. Therefore, the skin is normally abundantly innervated by nociceptor nerve terminals.

Methods

Preparation of DETA/Poly Ornathine/Laminin/Fibronectin Surface:

The surface was prepared as described (Guo, X., et al., Biomaterials, 2013 34(18):4418-4427). Specifically, DETA glass coverslips (Stenger, D. A., et al., Brain Research, 1993. 630(1-2):136-147) were soaked overnight in 15 μg/ml Poly-ornithine diluted in PBS. Next day, these coverslips were washed briefly with PBS and then coated with 3.3 μg/ml laminin and 10 μg/ml fibronectin in PBS for a period of at least 2 h (usually overnight). Right before cell plating, the solution was removed and the surface was allowed to dry for a quick period (<5 min).

Induction of Nociceptor Neurons from hNP1 Cells:

Human neural progenitor cells, STEMEZ™hNP1, were obtained from Neuromics (Edina, Minn.). In the present study, passage 9 or 10 cells were used and the induction procedure consisted of three steps. For proliferation, 1×10⁶ cells were seeded into a 35 mm cell culture dish which was pre-coated with BD Matrigel® (BD Biosciences, cat. 356234, 1:200 diluted in DMEM, 1 hour at room temperature), and maintained in the proliferation medium (supplemented AB2™ basal medium from Neuromics containing 20 ng/ml bFGF (R&D system, cat. 234-FSE-025/CF); the medium was changed every other day. The cells were proliferated for 3 to 5 days until 100% confluence was reached. Next, they were manually dissociated, re-plated onto glass coverslips pre-coated with DETA/Poly-ornathine/Laminin/Fibronectin at a density of 400 cells/mm². The cells were expanded in proliferation medium for 2 to 3 days to enable ˜90% confluence before induction. To initiate sensory neuron differentiation, the medium was replaced with KSR medium that contained 10 μM SB43152 and 100 nM LDN-193189 (Counted as day 0).

KSR medium was prepared by supplementing 800 ml Knockout DMEM (Invitrogen, Cat. 11330-032) with 150 ml KSR (knockout serum replacement, (Invitrogen, Cat. 10828-028), 10 ml L-Glutamine (Invitrogen, Cat. 21051-016), 10 ml Penicillin/Streptomycin (100×, Invitrogen, Cat. 15070-063), 10 ml 10 mM MEM (100×, nonessential amino acids, Invitrogen, Cat. 11140-050) and 1 ml β-mercaptoethanol (1,000×, Invitrogen, Cat. 21985-023). On day 2, the cells were fed with KSR medium containing four small molecules (100 nM LDN-193189, 8 μM SB431542, 3 μM CHIR99021, 4 μM DAPT). Then from day 4 and on to feed the cells during differentiation, the medium was replaced and gradually switched from KSR medium to N2B medium (NeuralStem Inc) according to the following schedule: day 4 (75% KSR, 25% N2B), day 6 (50% KSR, 50% N2B), day 8 (25% KSR, 75% N2B), days 10 (0% KSR, 100% N2B). However, the content of the four small molecules remained constant throughout the procedure. Starting with day 12, the cells were fed with a differentiation medium by changing ½ of the medium every 2 days. The differentiation medium consisted of N2B medium supplemented with BDNF (Cell Sci. cat. CRB600B, 25 ng/ml), GDNF (Cell Sci. Cat. CRG400B, 25 ng/ml), human-b-NGF (R&D systems, cat. 256-GF, 25 ng/ml), The cells were analyzed by immunocytochemistry and electrophysiology starting from day 14.

Co-Culture of Nociceptors with Human Skin Constructs:

The skin construct included skin-like cells derived from induced pluripotent stem cells. The hydrogel was prepared as a mixture of collagen I (1.5 mg/ml, Gibco Cat No. A10483) and Matrigel (1 mg/ml, BD Matrigel) diluted in DPBS. Differentiated human nociceptors were harvested with trypsin and added into the hydrogel at a density of 1.5×10⁶ cells/ml. The hydrogel with cells was then plated into the millicell cell culture insert (Millipore, 1 μm or 0.4 μm) which was placed in a well of a 12-well plate. The hydrogel was allowed to gel in 37° C. incubator. The human skin construct was placed on top of the hydrogel right before the end of the gelation period so that the bottom part of the skin was merged in the hydrogel while the upper part could still be exposed to the air. After the completion of geltation, the well was filled with serum-free differentiation medium up to the level where half of the skin was submerged. The co-culture was maintained in the incubator (37° C., 5% CO₂) and fed every 2 days by changing half of the medium.

Results

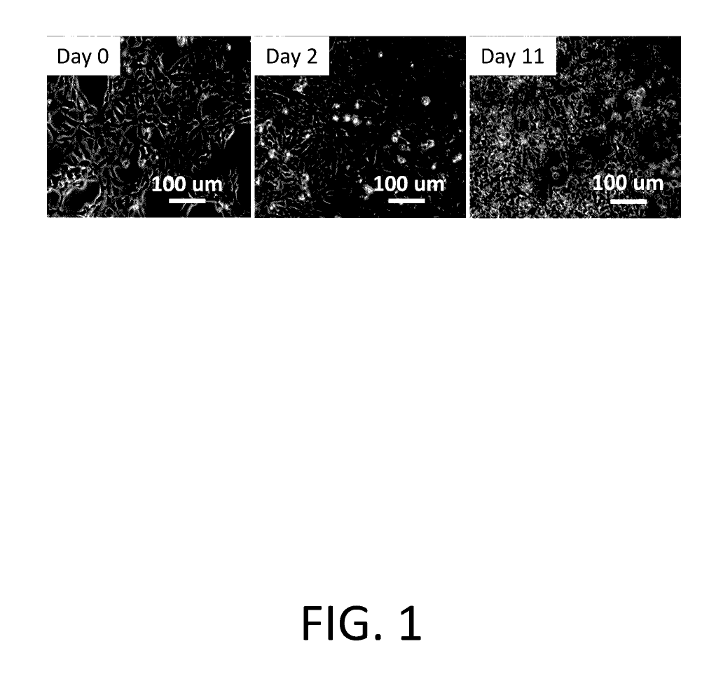

A protocol was first developed to induce nociceptors from human neural progenitors. FIG. 1 shows phase contrast pictures demonstrating the morphological change of the cells at days 0, 2, and 11 of the nociceptor induction.

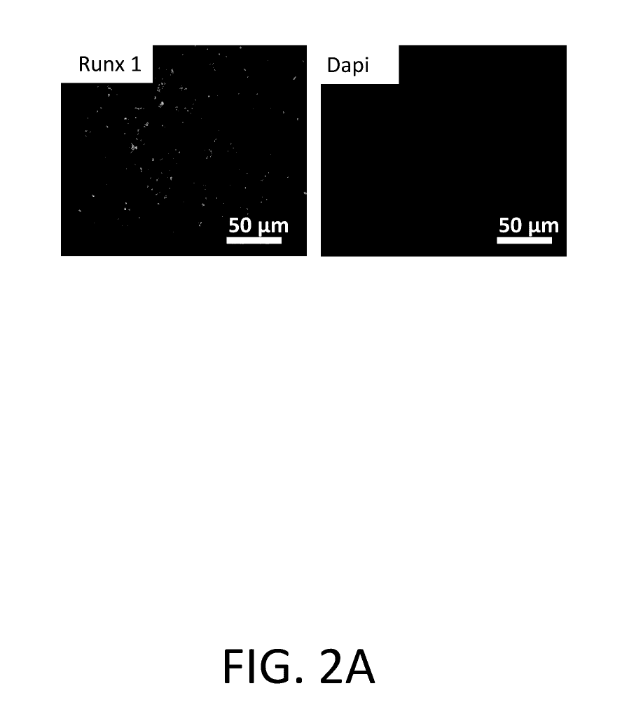

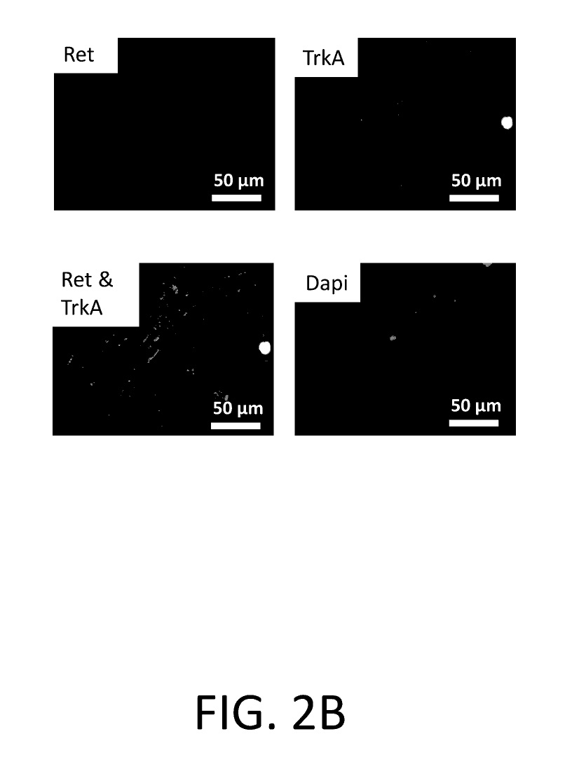

The identity of induced nociceptors was analyzed by nociceptor-specific markers Runx1, TrkA, Ret, IB4, CGRP as well as peripheral neuron marker Peripherin. FIG. 2 shows immunocytochemistry characterization of differentiated cells indicated the expression of typical nociceptor markers. FIG. 2A shows Runx1 and DAPI staining in a Day 7 culture. The images are taken from the same area of the culture plate/same field of view. FIG. 2B shows co-immunostaining of Ret and TrkA in a Day 13 culture. FIG. 2C shows co-immunostaining of TrkA and Peripherin in a Day 28 culture.

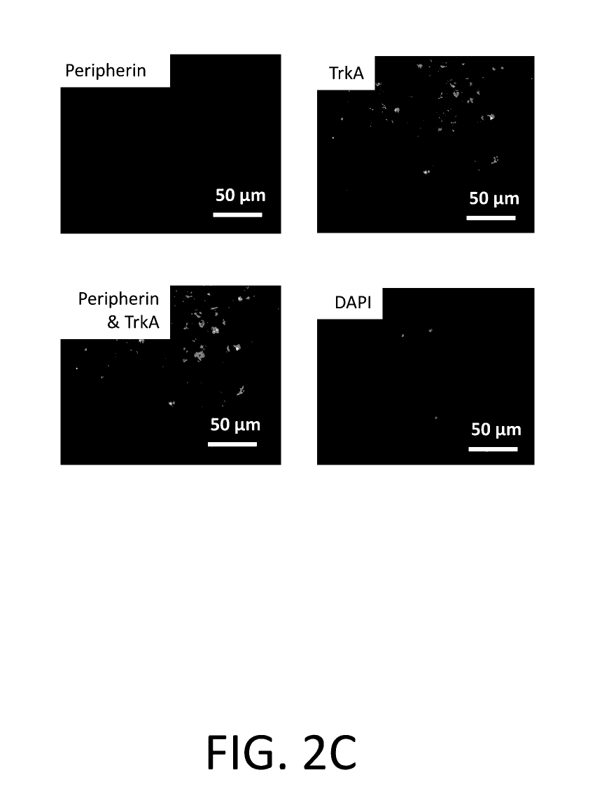

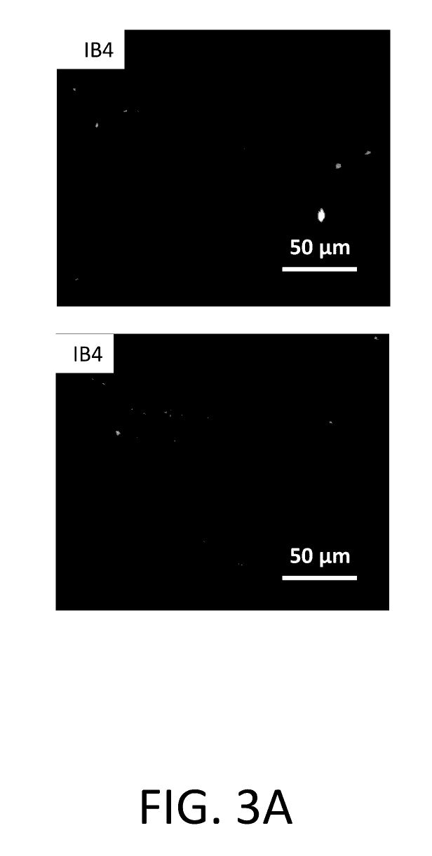

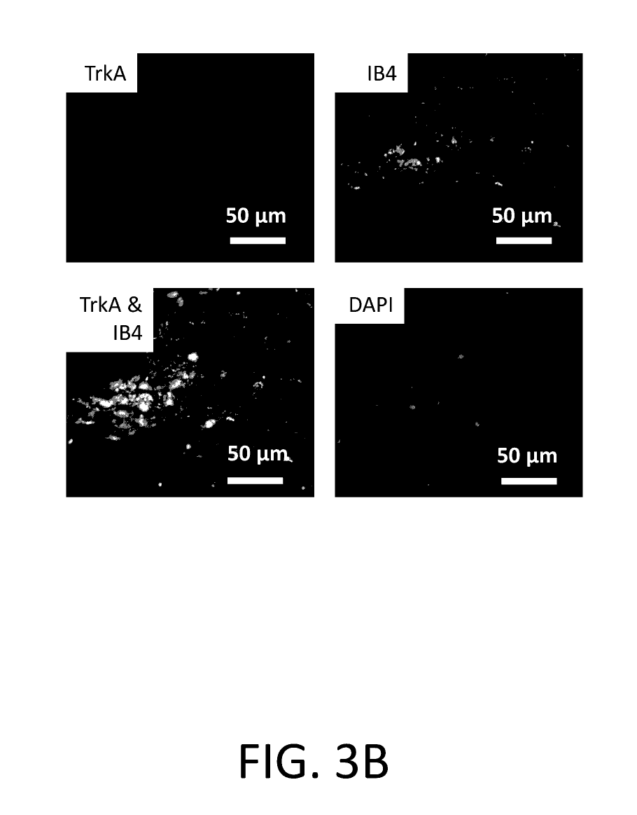

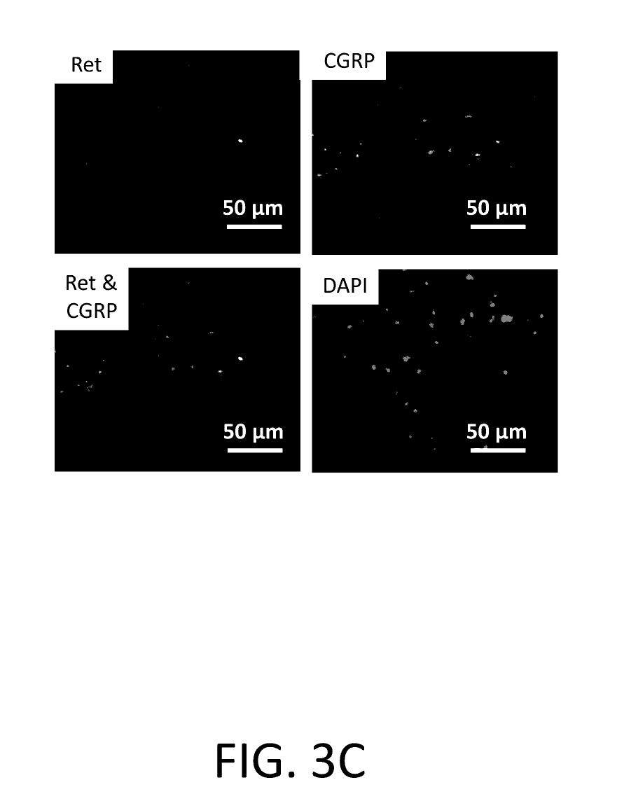

FIG. 3 shows characterization of induced nociceptors with peptidergic marker CGRP and non-peptidergic marker IB4. FIG. 3A shows IB4 staining at Day 24. FIG. 3B shows co-immunostaining of IB4 and TrkA at Day 47. FIG. 3C shows co-immunostaining of CGRP and Ret at Day 30.

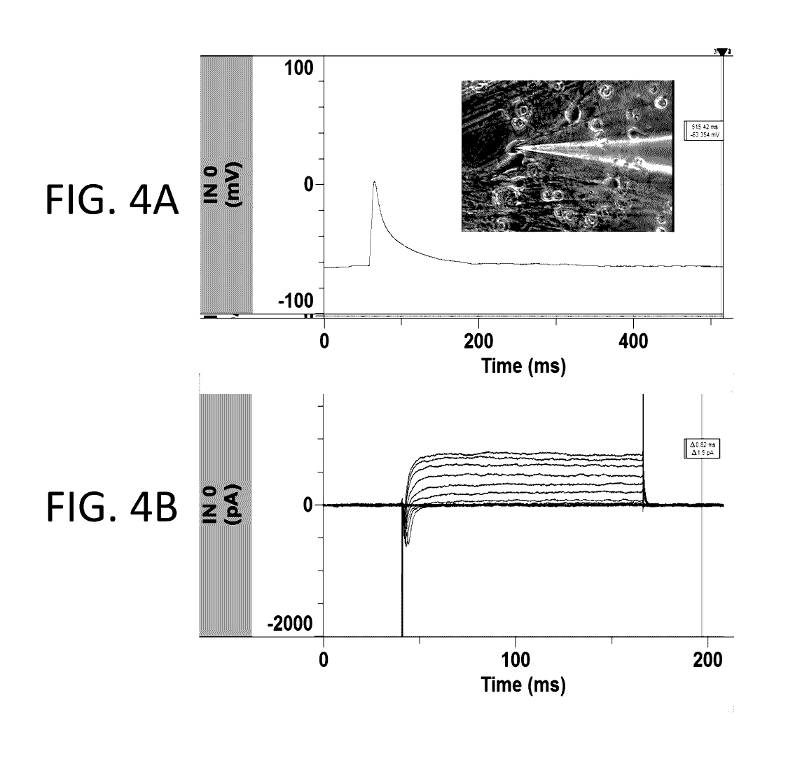

Patch clamp recording confirmed the functionality of induced neurons. FIG. 4 shows electrophysiological recording from an induced culture. FIG. 4A shows an action potential recorded from an induced neuron shown as in the inset picture. FIG. 4B shows inward (INa+) and outward (IK+) currents recorded by voltage-clamp.

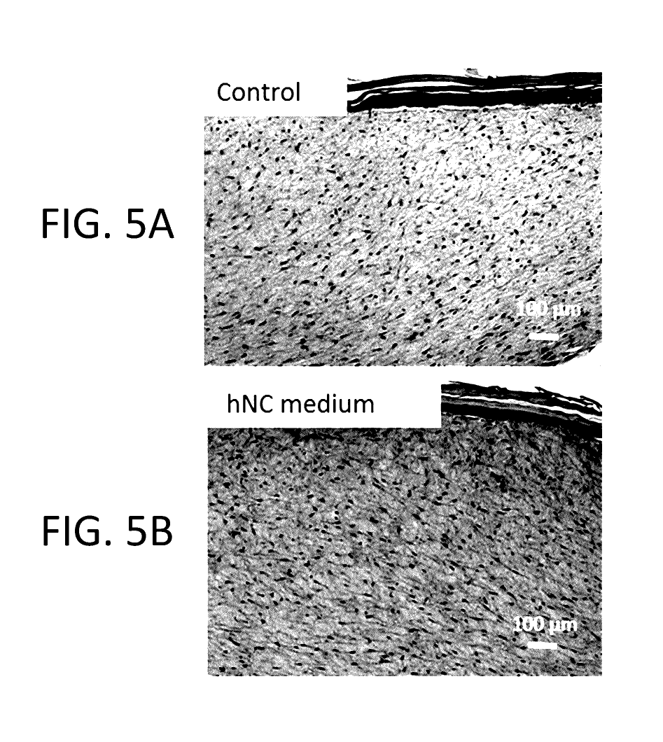

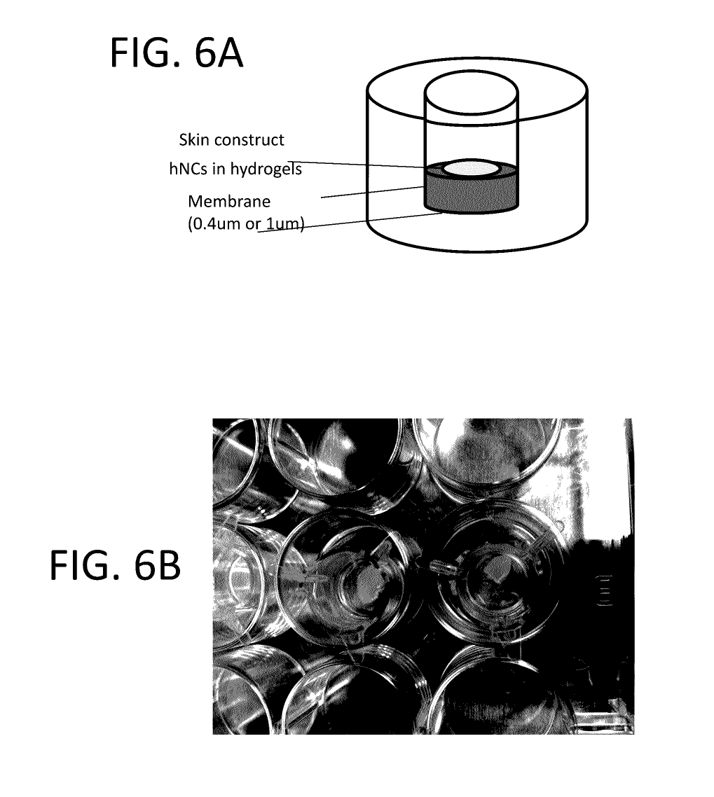

These nociceptors were then co-cultured with human iPSC-derived skin construct. A serum-free co-culture medium was tested to ensure the viability of both types of cells. FIG. 5 shows H & E staining of the human skin construct after one week of culture in a serum-containing control skin-maintenance medium (FIG. 5A) and serum-free human nociceptor medium (FIG. 5B). To emulate in vivo skin innervation situation, a 3D trans-well system was then developed for the co-culture in which nociceptors were plated in hydrogel inside a trans-well membrane insert (either a 0.4 micron or a 1 micron membrane) and the skin construct was placed on top and cultured under a semi-air condition. FIG. 6A shows a diagram of this setup, and FIG. 6B shows a picture of the co-culture setup.

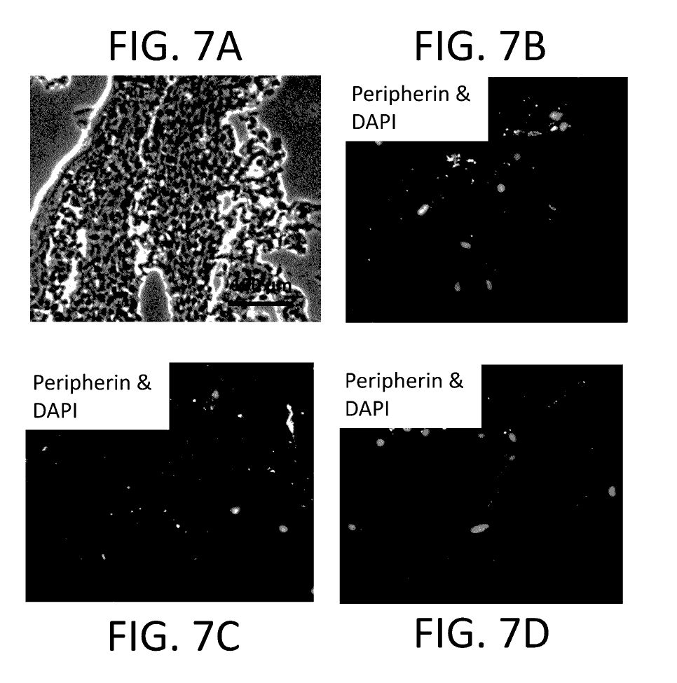

Immunostaining of the skin sections with Peripherin after nineteen days co-culture displayed the penetration of sensory axons into the skin tissue. FIG. 7A shows H&E staining. FIGS. 7B-C show immunohistological staining of the skin sections demonstrated the penetration of sensory neuron axons (stained by Peripherin) into the skin.

What is claimed is:

1. A method of differentiating human neural progenitor cells to nociceptor-like cells, comprising:
a) exposing human neural progenitor cells to a first serum-free initiation medium for a first time period; and
b) exposing the human neural progenitor cells to a second serum-free initiation medium for a second time period; and
c) exposing the human neural progenitor cells to a serum-free differentiation medium during a third time period to cause at least a portion of the human neural progenitor cells to differentiate into nociceptor-like cells;
wherein the nociceptor-like cells possess at least one nociceptor-like property.

2. The method of claim 1, wherein the first serum-free initiation medium comprises KSR base medium, SB43152, and LDN-193189.

3. The method of claim 2, wherein the concentration of SB43152 in the first serum-free initiation medium is from 1-100 μM, and wherein the concentration of LDN-193189 in the first-serum free initiation medium is from 10-1000 nM.

4. The method of claim 3, wherein the SB43152 concentration in the first-serum free initiation medium is about 10 μM, and wherein the concentration of LDN-193189 in the first-serum free initiation medium is about 100 nM.

5. The method of claim 1, wherein the first time period is from 1-5 days.

6. The method of claim 5, wherein the first time period is about 2 days.

7. The method of claim 1, wherein the second serum-free initiation medium comprises SB43152, LDN-193189, CHIR99021, and DAPT.

8. The method of claim 7, wherein the concentration of SB43152 in the second serum-free initiation medium is from 0.8-80 μM, the concentration of LDN-193189 in the second serum-free initiation medium is from 10-1000 nM, the concentration of CHIR99021 in the second serum-free initiation medium from 0.3-30 μM, and wherein the concentration of DAPT in the second serum free initiation medium is from 0.4-40 μM.

9. The method of claim 8, wherein the concentration of SB43152 in the second serum-free initiation medium is about 8 μM, the concentration of LDN-193189 in the second serum-free initiation medium is about 100 nM, the concentration of CHIR99021 in the second serum-free initiation medium about 3 μM, and wherein the concentration of DAPT in the second serum free initiation medium is about 4 μM.

10. The method of claim 7, wherein the second serum-free initiation medium further comprises KSR base medium, N2B base medium, or a combination thereof.

11. The method of claim 10, further comprising gradually reducing a ratio of KSR base medium to N2B base medium during the second time period.

12. The method of claim 1, wherein the second time period is from 9-15 days.

13. The method of claim 12, wherein the second time period is about 10 days.

14. The method of claim 1, wherein the serum-free differentiation medium comprises N2B base medium, BDNF, GDNF, and NGF.

15. The method of claim 14, wherein the concentration of BDNF in the serum-free differentiation medium is from 2.5-250 ng/mL, the concentration of GDNF in the serum-free differentiation medium is from 2.5-250 ng/mL, and the concentration of NGF in the serum-free differentiation medium is from 2.5-250 ng/mL.

16. The method of claim 15, wherein the concentration of BDNF in the serum-free differentiation medium is about 25 ng/mL, the concentration of GDNF in the serum-free differentiation medium is about 25 ng/mL, and the concentration of NGF in the serum-free differentiation medium is about 25 ng/mL.

17. The method of claim 1, wherein the third time period is at least two days.

18. The method of claim 1, wherein the human neural progenitor cells are in contact with an aminated alkylsilane during the first time period, the second time period, the third time period, or any combination of the above.

19. The method of claim 18, wherein the aminated alkylsilane is DETA.

20. The method of claim 1, wherein the nociceptor-like property comprises expression of Runx1, TrkA, Ret, IB4, CGRP or a combination thereof.

21. The method of claim 1, wherein the nociceptor-like property comprises expression of a mRNA transcript partially or fully encoding Runx1, TrkA, Ret, IB4, CGRP, or a combination thereof.

22. A method of making an innervated tissue construct, the method comprising,
a) differentiating human neural progenitor cells to nociceptor-like cells according to the method of claim 1;
b) contacting the nociceptor-like cells with a tissue construct;
c) incubating the nociceptor-like cells and the tissue construct under conditions suitable to induce axon extension from the nociceptor-like cells into the tissue construct.

23. The method of claim 22, further comprising suspending the nociceptor-like cells in a hydrogel.

24. An engineered construct for screening candidate treatments, the construct comprising:
a) a tissue construct;
b) nociceptor-like cells differentiated from human neural progenitor cells according to the method of claim 1, the nociceptor-like cells comprising axons;
c) wherein the axons extend from the nociceptor-like cells into the tissue construct.

25. The engineered construct of claim 24, wherein nociceptor-like cells are encapsulated in a hydrogel.

26. The engineered construct of claim 24, wherein the tissue construct is a skin construct comprising skin or skin-like cells.

27. A method of screening candidate treatments, comprising:
a) exposing the engineered construct of claim 24 to a candidate therapy, and
b) measuring a response from at least one nociceptor-like cell within the tissue construct.
